(19) United States
(12) Patent Application Publication
(10) Pub. No.: US 2005/0053682 A1
(43) Pub. Date: Mar. 10, 2005

(54) Oral treatment for anorectal conditions

(75) Inventors: Yi Zhang, Chengdu (CN); Yi Zhang, Chengdu (CN); Qingjiang Wu, Chengdu (CN)

Correspondence Address: SUGHRUE MION, PLLC, 2100 PENNSYLVANIA AVENUE, N.W., SUITE 800, WASHINGTON, DC, 20037, US

(73) Assignee: VITAL PHARMACEUTICALS(SICHUAN) COMPANY LIMITED

(21) Appl. No.: 10/658,194

(22) Filed: Sep. 10, 2003

(51) Int. Cl. A61K 35/78

(52) U.S. Cl. 424/775; 424/778

(57) ABSTRACT

Methods and uses pertaining to the oral treatment of anorectal conditions in a man or woman in need of such treatment are described, which comprises treatment with an orally effective amount of an extract of Cortex Dictamni in unit dosage form in association with one or more carriers or excipients.

This invention relates the oral treatment of anorectal conditions, including in particular haemorrhoids.

Haemorrhoids may be regarded as one of the most common ailments in human society, affecting both males and females. Haemorrhoids are more prevalent after the age of 30.

The exact cause or causes of haemorrhoids are unknown. However, the upright posture of humans forces a great deal of pressure on the rectal veins about the anus, which may cause them to bulge. Other contributing factors to haemorrhoid formation may include aging, chronic constipation or diarrhoea, pregnancy, hereditary disposition, faulty bowel function due to over use of laxatives or enemas or straining during bowel movements, or even spending unduly long periods of time in the process of defecation.

Symptoms of haemorrhoids include bleeding during bowel movements, protrusion of haemorrhoids during bowel movements, itching in the anal area, pain, sensitive lumps, and general anal discomfort.

The prevalence of haemorrhoids in the United States of America is reported to be in the order of 10.4 million people, with a prevalence rate of 3.82%. Reports of the annual incidence in the United States of new cases of haemorrhoids is 1 million cases per year.

Mild cases of haemorrioids may be relieved by increasing the amount of fibre and fluids in the diet, or eliminating excessive straining during defecation. Sits baths, that is sitting in plain warm water for about 10 minutes, may also provide some relief. Many cases of haemorrhoids, however, are not of a mild, easily resolvable nature. Such haemorrhoids require medical procedures carried out by colorectal surgeons, proctologists or gastroenterologists.

Current medical treatment for haemorrhoids includes rubber band ligation treatment. This treatment carried out on an outpatient basis works effectively on internal haemorrhoids, that is haemorrhoids which arise above the dentate line in the anal canal. A small rubber band is placed over the haemorrhoids, cutting off its blood supply. The procedure involves anal examination, insertion of a proctoscope into the anal canal, and banding of the haemorrhoid. The procedure involves anal discomfort, and patients may have difficulty urinating for up to 12 hours or more after the procedure. There may also be bleeding from the haemorrhoids which are banded. Apart from the discomfort of the process, banded internal haemorrhoids may reoccur and require further banding treatment.

Injection and coagulation may also be used on bleeding haemorrhoids that do not protrude from the anus. Again, these methods involve anorectal examination, and may cause anal pain and anal bleeding.

Haemorrhoidectomy, that is surgery to remove haemorrhoids, is generally regarded as the best method for treatment of haemorrhoids. It is necessary when clots repeatedly form in external haemorrhoids, ligation fails to treat internal haemorrhoids, protruding haemorrhoids can not be reduced, or persistent bleeding remains. A haemorrhoidectomy is conducted under anaesthesia, with the surgeon removing excessive tissue which causes the bleeding and haemorrhoid protrusion. The procedure requires hospitalisation, and a period of inactivity and convalescence. The wounds created by surgery are painful, and bowel movements following surgery may be so painful as to require analgesics. Other treatments for haemorrhoids include cryotherapy and application of direct current. Cryotherapy, which was popular many years ago, consists of freezing haemorrhoidal tissue, which drops off following the freezing process. The procedure is regarded as being very painful, and has generally fallen out of favour. Direct current methods shrink haemorrhoids, but have not gained wide acceptance due to questions over its effectiveness, and the uncomfortable nature of the treatment.

A number of topical compositions for application to haemorrhoids of the anal canal have been proposed and are sold over the counter in pharmacies and drug stores. Compositions containing anti-inflammatory agents and topical anaesthetics may be available on prescription. Whilst such compositions may provide some mild symptomatic relief from the symptoms of haemorrhoids, they are messy to apply and may be regarded as ineffective in the treatment of haemorrhoids.

An anal fissure is a small tear or cut in the anal canal which causes acute pain and/or bleeding. The pain associated with anal fissure is reported to be so extreme or severe that patients may seek to avoid defecation due to the pain experienced.

Anal fissures may be acute (of recent onset) or chronic (present for a long time or occurring frequently). Chronic anal fissures, or acute anal fissures which do not resolve over a period of a matter of weeks, generally require surgical intervention, known as a sphincterotomy. Surgery involves a surgeon cutting a portion of the internal sphincter muscle, which relieves tension or spasm in the anal sphincter. The sphincterotomy may carried out as an outpatient procedure, or in a hospital stay. The surgery to cut the anal sphincter may be painful, and give rise to anal incontinence.

Anal pruritus, also known as pruritus ani is a common condition. It may be characterised as in irresistible urge to scratch the anal area, or general anal pain. Anal pruritus may be particularly troublesome or following bowel movements. The factors which contribute to pruritus ani are unclear and may include haemorrhoids, anal fissure or other causes.

One or more symptoms of anal bleeding, anal pain, anal irritation, irritating mucous discharge, anal inflammation and constipation may be associated with anorectal conditions such as haemorrhoids, anal fissure and anal pruritus.

Unexpectedly, it has now been found that the herbal extract of Cortex Dictamni is useful in the treatment of anorectal conditions. Furthermore, the compositions of the invention may be administered orally, thereby obviating the disadvantages associated with topical anorectal application, and colorectal surgical procedures.

Thus, the invention concerns in one aspect a method of treating anorectal conditions in a man or woman, comprising orally administering to a patient in need of such treatment an orally effective amount of an extract of Cortex Dictamni in unit dosage form in association with one or more carriers or excipients.

The term Cortex Dictamni refers to the root bark of plants of the genus Dictamnus dasycarpus Turcz (family Rutacae). Cortex Dictamni may also be known by the common name Densefruit Pittany Root Bark. Cortex Dictamni is described in the Pharmacopoeia of the People's Republic of China, 1995 edition, published by Guandong Science and Technology Press and Chinese Materia Medica Dictionary, 1977 edition, published by Jiangsu New Medical College. The Chinese Materia Medica Dictionary describes the use of Cortex Dictamni in skin inflammation, eczema, scabies and tinea. The dried root bark is decocted, or ground into a powder, and used topically for the treatment of various skin conditions. Applicants have now surprisingly found that extracts of Cortex Dictamni may be used to orally treat anorectal conditions, such as haemorrhoids.

Dictamnus dasycarpus Turcz is a perennial plant which thrives on hillsides in jungles. It is particularly grown in China in Dongbei, Hebei, Shandong, Henan, Anhui, Jiangsu, Jiangxi, Sichuan, Guizhou, Shanxi, Gangsu and in Inner Mongolia.

This plant is collected in spring and autumn in northern China, and collected in the summer in southern China After harvest, the bark of the root is removed and air dried. The dried root-bark is of a rolled shape or double-rolled shape, 7-12 centimeters long, with a diameter of 1-2 centimeter, 2-5 millimeter thick. The surface colour varies from yellow-white to maple, slightly smooth. Sometimes there are lengthways wrinkles and side root marks. The inner surface has a straw yellow colour; it is smooth and shapes into a round cylinder by the side root. The material is crispy and easy to break. The fracture side is in layers with an ivory colour. White colour fine crystals can be seen under daylight or lamp.

Preferably, the Cortex Dictamni for oral administration is provided as an extract such as a chemical or physical extract of Cortex Dictamni. Chemical extracts of Cortex Dictamni may be prepared by organic solvent extraction of Cortex Dictamni, followed by solvent removal. On solvent removal, such as by evaporation or distillation, the residue may be further dried as necessary and mixed with one or more carriers or excipients. The extract may be in powdered form following solvent removal and drying, and may be further ground to a fine particulate form and mixed with one or more excipients. Organic solvents used to extract Cortex Dictamni include ethanol, propanol, chloroform, methanol, ether, acetone and benzene. C₁-C₈ organic solvents are preferred. Cortex Dictamni may be extracted by a mixture of water and/or organic solvent, such as C₁-C₈ organic solvent, for example ethanol. The amount of organic solvent, such as ethanol in the water/organic solvent mix may be from 25 to 95%, preferably from 50 to 95% volume/volume. On removal of solvent, such as by evaporation or distillation, the residue may be further dried as necessary and mixed with one or more carriers or excipients. Extracts may also be prepared by CO₂ cryogenic extraction, such as supercritical fluid extraction and low pressure cold extraction. Other well known herbal extraction procedures may be used, including kinetic maceration and percolation. Extracts of Cortex Dictamni may also be prepared by physical processes, such as grinding the dried bark into a fine particulate form.

Anorectal conditions which be may treated according to the present invention include haemorrhoids (both internal haemorrhoids, external haemorrhoids and mixed internal and external haemorrhoids), chronic and acute anal fissure, anal pruritus and constipation. Symptoms associated with one or more anorectal conditions may also be treated in accordance with the present invention, these including one or more of anal bleeding, anal pain, anal irritation, mucous discharge causing anal irritation, faecal discharge, constipation and anal inflammation.

Orally effective amounts of extracts of Cortex Dictamni for oral administration comprise 5 mg to 50 g of extract. Preferably unit dosage forms, such as tablets or capsules, for example gelatine or other capsules, may contain from 50 to 800 mg Cortex Dictamni extract. By way of example an average daily dose for a 60 kg person is 0.32-5.76 g of extract. Treatment may be from two to ten days, or longer. Generally, treatment of conditions or symptoms thereof are noticed after two days.

Pharmaceutically acceptable carriers and/or excipients are mixed or blended with the Cortex Dictamni extract. Conventional pharmaceutically acceptable carriers and excipients well known in the art may be used, including for example agar, alginic acid, calcium alginate, calcium carbonate, calcium sulphate, carboxymethyl celluloses, compressible sugar, confectioner's sugar, dextrates, dextrin, dextrose, dicalcium phosphates, fructose, gelatin, glyceryl palmitostearate, guar gum, hydroxyethyl cellulose, hydroxymethyl cellulose, hydroxypropyl cellulose, methyl cellulose, lactose, magnesium carbonate, magnesium oxide, magnesium stearate, maltitol, maltodextrin, mannitol, microcrystalline cellulose, polymethacylates, potassium chloride, powdered cellulose, pregelatinized starch, sodium alginates, sodium aluminium silicates, sodium chloride, sorbitol, starch, sodium starch glycoate, starch sterilizable maize, sucrose, sugar spheres, and tribasic calcium phosphates Two or more excipients may be used. Extracts may be formed into capsules, such as gelatine capsules or other standard capsules (such as soft or hard gel capsules) used in the pharmaceutical field. Extracts may be tabletted with standard excipients and carriers as are well known in the art.

Without expressly limiting the invention and the mechanism of action, it is believed by the applicant that the active components in the Cortex Dictamni extract comprise one or more compounds selected from dictamine, obacunone and fraxinellone. Each of these compounds is currently believed to have activity in the treatment of anorectal conditions. These compounds have the following formulas:

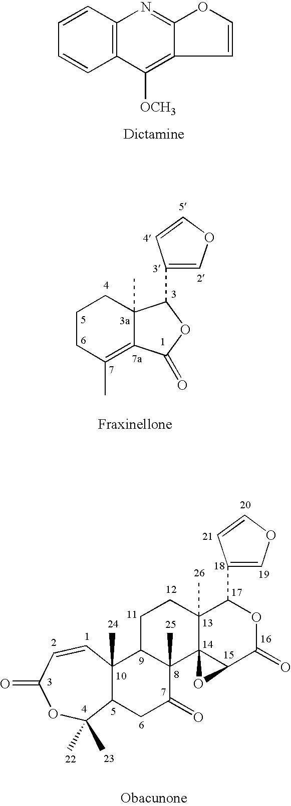

Dictamine, obacunone and fraxinellone are purified from organic solvent extracts particularly ethanol extracts of Cortex Dictamni, which are subject to chromatography, such as high performance liquid chromatography (HPLC), to isolate the individual compounds, followed by recrystallisation. In the alternative, the compounds may be chemically synthesised according to methods known in the art. For example, dictamine may synthesised according to the methods set out by Tuppy et al, Angew. Chem. 68, 388 (1956).

In accordance with another aspect of this invention there is provided a method of treating anorectal conditions in a man or woman, comprising orally administering to a patient in need of such treatment with an orally effective amount of one or more compounds selected from dictamine, obacunone and fraxinellone, optionally in association with one or more pharmaceutically carriers or excipients.

A therapeutically effective amount of the one or more compounds dictamine, obacunone and fraxinellone is believed to be in the range of 0.5 to 150 mg per day. An average daily dose of extract for a 60 kg person of 0.32 to 5.76 g corresponds to about 0.8 to 15 mg dictamine.

The invention also provides use of an extract of Cortex Dictamni for the manufacture of an orally deliverable medicament in unit dosage form for the treatment of anorectal conditions.

In another aspect the invention relates to use of an extract of Cortex Dictamni in the manufacture of an orally administrable medicament for the relief of symptoms of anorectal disease selected from one or more of anal bleeding, anal pain, anal irritation, mucous discharge from the anus, constipation and anal inflammation.

In another aspect of this invention there is provided an orally administrable composition in unit dosage form comprising an extract of Cortex Dictamni in association with one or more pharmaceutically carriers or excipients for the treatment of anorectal conditions.

Prior to the invention of the applicant, oral treatment of anorectal conditions or anorectal disease, or treatment of the symptoms of anorectal disease has not been available. The finding by the applicant that a single herbal extract from Corpus dictamni in unit dosage form may be used to orally treat anorectal conditions, and the symptoms of anorectal conditions and disease, is particularly advantageous in this regard. The use of a single herbal extract is particularly advantageous with regard to simplicity of preparation of the compositions of the invention, efficacious results, standardisation and reproducibility.

Extracts of Corpus dictamni may additionally be used with extracts of one or more other herbs such as Flos Sophore, Radix Sophora Flavescentis, Radix Amepelopsis, Radix Rubiae, Radix Sanguisorbae, Cortex Phellodendri, Radix Arnebie, Radix Scutellaria, and Rhizoma Coptidis. Where additional herbal extracts are combined with the extract of Cortex Dictamni, said herbs are present at a ratio from 1:1-10 Cortex Dictamni extract on a weight/weight basis. Additional herbal extracts are prepared in the same manner as for Cortex Dictamni.

Extracts of Cortex Dictamni may be particularly used in association with extracts of Radix Sophora Flavescentis, at a ratio of 1-10:1, particularly 3:1 w/w.

The prior art has not recognised that extracts Cortex Dictamni alone may be used for the oral treatment of anorectal conditions, such as haemorrhoids. Nor has the prior art recognised that extracts of Cortex Dictamni may be used to treat the symptoms of anorectal conditions such as bleeding, anal pain, anal irritation, mucous discharge from the anus, faecal discharge and anal inflammation.

Embodiments of the invention will now be described with reference to the following non-limiting examples.

### EXAMPLE 1

### Preparation of Cortex Dictamni Extracts

Murine model for Treatment of Anorectal Disease

A mouse model was established to investigate activity of Cortex Dictamni extracts. In this model local anal irritation was induced by acetic acid application to the anus of the mice. Oral administration of extracts twice per day to the acetic acid treated mice was used as a preliminary model for activity of extracts. In the same mouse model extracts of Cortex Dictamni were tested for pain desensitistion activity in a “hot plate” assay where prolongation of the time of reaction to pain caused by stimulating the mice feet by heat was determined.

From these studies it was determined that ground extracts of Cortex Dictamni were effective, water extracts of Cortex Dictamni were ineffective and organic solvent or organic solvent/water extracts of Cortex Dictamni were effective in the mouse assay model.

C₁-C₈ organic solvents were found to be particularly suitable for preparing extracts of Cortex Dictamni. Organic solvents tested included ethanol, indole, acetic acid, glycerine, chloroform, ether, light petroleum and propylene glycol. The efficacy of active component extract from Cortex Dictamni was generally proportional to organic solvent concentration. In extracting mixtures of water and organic solvent, it was generally found that at least 20% organic solvent was required to extract active components. In one study water/ethanol extracting mixtures were tested comprising 95% ethanol, 75% ethanol, 55% ethanol, 35% ethanol and 10% ethanol. This study showed that the greater the concentration of organic solvent the greater the amount of active components was extracted in the organic solvent fraction.

5 kg of Cortex Dictamni was extracted with 30 litres of 95% ethanol (at a ratio of 6 parts extracting solvent to 1 part Cortex Dictamni). After 60 minutes the solvent extract was recovered, and the remnant material was again extracted with 6 volumes of organic solvent. The combined solvent extracts were filtered, and then concentrated by removing ethanol under vacuum to give a dried paste. The dried paste was ground into a fine powder and mixed with corn starch and talc. Typical capsules contained 5-30% excipients with the remainder incorporating extract. The resulting extract is used to fill hard gel capsules.

Each capsule contains 0.42 g of extract.

Extracts of Cortex Dictamni and Radix Sophora Flavescentis at a ratio of 3:1 was prepared as follows:

3.75 kg Cortex Dictamni and 1.25 kg Radix Sophora Flavescentis are extracted with 6 volumes of 85% ethanol for 1 hour and the extraction procedure repeated. After filtration, the filtrated organic solvent was concentrated by ethanol removal and vacuumising to give a dry paste. The dry paste was ground into a fine powder, and corn starch and talc was added. The extract was then used to fill hard gel capsules.

### Example 2

### Clinical Studies

30 patients were studied, comprising 19 males and 11 females suffering from various anorectal disorders including haemorrhoids, anal pain and inflammation, anal pruritus, constipation, swollen haemorrhoids and bloody stool (associated with anal fissure). Patients were administered two capsules of 320 mg of Cortex Dictamni for oral administration each day over 2 day period following diagnosis. Results of this study are set out in Table 1.

| | Total | | % | Improve- | % Im- | No | % No | |
|---|---|---|---|---|---|---|---|---|
| Symptoms | Cases | Recovery | Recovery | ment | provement | effect | effect | Efficacy |
| Bloody stool (Hemotochezia) | 25 | 10 | 40 | 13 | 52 | 2 | 8 | 92 |
| Bleeding during or after bowel | 13 | 7 | 54 | 6 | 46 | 0 | 0 | 100 |
| movements | | | | | | | | |
| Haemorrhoids protrude during | 13 | 5 | 38 | 4 | 31 | 4 | 31 | 69 |
| bowel movements | | | | | | | | |
| Anal pain and swelling | 5 | 2 | 40 | 2 | 40 | 1 | 20 | 80 |
| Anal swelling | 16 | 1 | 6 | 11 | 69 | 4 | 25 | 75 |
| Anal pruritus | 9 | 1 | 11 | 6 | 67 | 2 | 22 | 78 |
| Constipation | 12 | 7 | 58 | 5 | 42 | 0 | 0 | 100 |
| Swollen haemorrhoid vessels | 30 | 4 | 13 | 20 | 67 | 6 | 20 | 80 |
| mucosa | | | | | | | | |
| Mucosal erosion of haemorrhoidal | 7 | 3 | 43 | 2 | 29 | 2 | 29 | 71 |
| zone | | | | | | | | |

The above study shows that treatment of the anorectal disease was observed after 2 days. Patients experienced amelioration or relief of the symptoms of the various anorectal diseases. Treatment continued for a further 7 days after the 2 day observation stage with patients experiencing continuing relief from their anorectal conditions.

In one specific study, a male aged 42 with protruding haemorrhoids following bowel movements, blood stained stools and occasional bleeding during bowel movements was studied. Initial diagnosis concluded internal haemorrhoids (phase 3), external haemorrhoids, and the initial finding of the treating doctor was that haemorrhoidectomy was required. Due to a hospital bed not being available, the patient was treated with Cortex Dictamni extract referred to above. The patient received 2 capsules per day for a 2 day period. Examination after 2 days showed reduction in haemorrhoid size and pain relief. The patient continued a further 4 days of treatment with the Cortex Dictamni extract with the patient experiencing relief of symptoms, and resolution of haemorrhoids, such that surgery to remove haemorrhoids was unnecessary.

In a further study patients suffering from various anorectal diseases/symptoms were treated with a composition comprising an extract of Corpus dictamni and extract of Radix Sophora Flavescentis at a ratio of 3:1. In this study 50 patients suffering variously from haemorrhoids, anal bleeding, anal pain and constipation were treated. Treatment was 2 capsules per day taken orally over a 2 day period. 24 cases clinically recovered with complete resolution of disease/symptoms, 16 cases showed a significant treatment of the symptoms of the disease, and in particular haemorrhoid size reduction, and 9 cases reported a positive effect, including pain relief and ease of defecation and arrest of bleeding. Positive effects were seen in 98% of the subject treated. This study demonstrates consistent results of patient improvement in relation to patients of different age and different anorectal conditions. In particular the study showed a remarkable result in arresting bleeding from the anus, pain relief and relief from constipation.

The above studies indicate that the oral treatment of haemorrhoids with extracts of Cortex Dictamni are effective for the treatment of anorectal conditions, particularly haemorrhoids, free of undesirable side effects.

### Example 3

### Isolation of Active Agents from Cortex Dictamni

5 kg of Cortex Dictamni was extracted with 6 parts of 95% ethanol, with extraction taking place for 1 hour at ambient temperature. This procedure was repeated 3 times, and the ethanol extracts pooled. Ethanol was removed and the extract brought to dryness by vacuum evaporation A yellow powdered concentrate resulted comprising approximately 1 kg of material. This extract was then chloroform extracted 3 times with 1 litre of chloroform. The chloroform extracts were pooled, and the chloroform removed under vacuum to give about 120 g of a red-brown oily liquid. The chloroform extract was then passed through a 300 micron silicon gel column. Fractions were then eluted with a gradient of petroleum ether and ethyl acetate. 500 ml fractions were collected and tested via thin layer chromatography. Fractions 16 to 21 were found to principally contain dictamine, fractions 8 to 15 were found to principally contain fraxinellone, and fractions 32 to 40 were found to principally contain obacunone. The fractions were crystallised from acetone, resulting in 520 mg of dictamine, 520 mg of fraxinellone, and 540 mg of obacunone. Compound identification was carried out via HPLC, IR, UV and NMR analysis.

The above compounds, and in particular dictamine, were considered to be the active agents in the extracts of Cortex Dictamni.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The reference to any prior art in this specification is not, and should not be taken as an acknowledgment or any form of suggestion that that prior art forms part of the common general knowledge in Australia.

1. A method of treating anorectal conditions in a man or woman, comprising administering to a patient in need of such treatment an orally effective amount of an extract of Cortex Dictamni in unit dosage form in association with one or more carriers or excipients.

2. The method of claim 1, wherein said one or more anorectal conditions are selected from haemorrhoids, anal fissure, anal pruritus, anal pain and constipation.

3. The method of claim 1, which is a method for the treatment of symptoms of anorectal conditions selected from one or more of bleeding, pain, anal irritation, mucus discharge, and anal inflammation.

4. The method of claim 2, wherein haemorrhoids are selected from internal haemorrhoids, external haemorrhoids, or a combination of internal and external haemorrhoids.

5. The method of claim 2, wherein anal fissure is selected from chronic and acute anal fissure.

6. The method of claim 1, wherein said extract is selected from C₁₋₈ organic solvent extract, a mixture of water/C₁₋₈ organic solvent extract, and ground or otherwise particulate Cortex Dictamni extract.

7. The method of claim 1, which additionally includes an extract of Sophora Flavescens.

8. The method of claim 7, wherein Sophora Flavescens extract is present at a ratio 1:1-10 Cortex Dictamni extract on weight/weight basis.

9. The method of claim 1, wherein said dosage is administered daily.

10. The method of claim 9, wherein said dosage is administered twice daily.

11. The method of claim 10, wherein said dosage comprises 5 to 150 mg of Cortex Dictamni extract.

12. The method of claim 1, wherein said extract comprises one or more of dictamine, fraxinellone and obacunone having the formula:

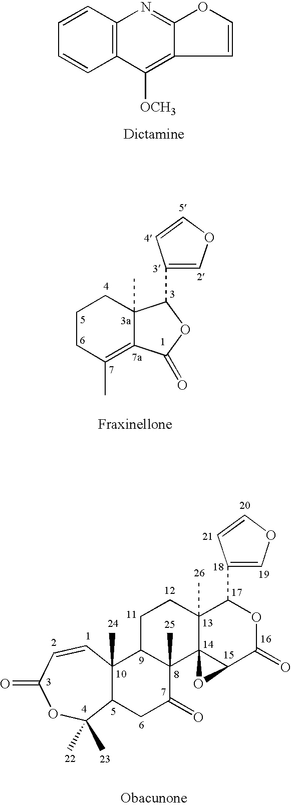

13. A method of orally treating a man or woman to ameliorate or relieve symptoms of one or more anorectal conditions, which comprises treatment with an orally effective amount of one or more compounds selected from the group consisting of dictamine, obacunone, and fraxinellone in association with one or more carriers or excipients.

14. An orally administrable composition in unit dosage form comprising an extract of Cortex Dictamni in association with one or more pharmaceutically acceptable carriers or excipients for the treatment of anorectal conditions.

15. The composition of claim 14, wherein said extract is selected from an organic solvent extract, a mixture of water/organic solvent extract, and ground or otherwise particulate extract of Cortex Dictamni.

16. The composition of claim 14, which additionally includes an extract of one or more of Flos Sophore, Radix Sophora Flavescentis, Radix Amepelopsis, Radix Rubiae, Radix Sanguisorbae, Cortex Phellodendri, Radix Arnebie, Radix Scutellaria, and Rhizoma Coptidis.

17. The composition of claim 14, wherein Sophora Flavescens extract is present at a ratio 1:1-10 Cortex Dictamni extract on weight/weight basis.

18. The composition of claim 14, wherein said dosage is administered daily.

19. The composition of claim 14, wherein said dosage is administered twice daily.

20. The composition of claim 14, wherein said dosage comprises 5 to 150 mg of Cortex Dictamni extract.

21. The method of claim 14, wherein said extract comprises one or more compounds selected from the group consisting of dictamine, obacunone and fraxinellone.

22. A composition for orally treating a man or woman to ameliorate or relieve symptoms of one or more anorectal conditions, which comprises treatment with an orally effective amount of one or more compounds selected from the group consisting of dictamine, obacunone and fraxinellone in association with one or more carriers or excipients.

23. (canceled)

24. (canceled)

25. A method according to claim 1 which additionally includes an extract of one or more of Flos Sophore, Radix Sophora Flavescentis, Radix Amepelopsis, Radix Rubiae, Radix Sanguisorbae, Cortex Phellodendri, Radix Arnebie, Radix Scutellaria, and Rhizoma Coptidis.
